(19) United States
(12) Patent Application Publication
(10) Pub. No.: US 2012/0215286 A1
(43) Pub. Date: Aug. 23, 2012

(54) Telemetry-Based Wake Up of an Implantable Medical Device in a Therapeutic Network

(75) Inventor: Md. Mizanur Rahman, Stevenson Ranch, CA (US)

(73) Assignee: Boston Scientific Neuromodulation Corporation, Valencia, CA (US)

(21) Appl. No.: 13/211,741

(22) Filed: Aug. 17, 2011

Related U.S. Application Data
(60) Provisional application No. 61/374,357, filed on Aug. 17, 2010.

(51) Int. Cl. A61N 1/36

(52) U.S. Cl. 607/60

(57) ABSTRACT

An external controller wishing to communicate with a particular microstimulator in a microstimulator therapeutic network broadcasts a unique wake-up signal corresponding to a particular one of the microstimulators. Each microstimulator has its unique wake-up signal stored in memory, and the wake-up signals for each microstimulator are also stored in the external controller. The microstimulators power up their receiver circuits to listen for a wake-up signal at the beginning of a power-on window. Each microstimulator not recognizing the received wake-up signal (because it does not match the wake-up signal stored in its memory) will power off their receivers at the end of the power-on window, or earlier once recognition cannot be established. The one microstimulator recognizing the received wake-up signal (because it matches the wake-up signal stored in its memory) will realize that the external controller wishes to communicate with it, and will send an acknowledgment to the external controller, which will in turn send the desired communication to the now-active microstimulator.

### CROSS REFERENCE TO RELATED APPLICATIONS

This application claims priority to U.S. Provisional Patent Application Ser. No. 61/374,357, filed Aug. 17, 2010.

### FIELD OF THE INVENTION

The present invention relates to a telemetry scheme for establishing communication between a plurality of implantable medical devices and an external component wishing to send data to one of the implantable medical devices.

### BACKGROUND

Implantable stimulation devices generate and deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, occipital nerve stimulators to treat migraine headaches, and other neural stimulators to treat urinary incontinence, sleep apnea, shoulder subluxation, etc. The present invention may find applicability in all such applications and in other implantable medical device systems, although the description that follows will generally focus on the use of the invention in a Bion® microstimulator device system of the type disclosed in U.S. patent application Ser. No. 12/425,505, filed Apr. 17, 2009.

Microstimulator devices typically comprise a small, generally-cylindrical housing which carries electrodes for producing a desired stimulation current. Devices of this type are implanted proximate to the target tissue to allow the stimulation current to stimulate the target tissue to provide therapy for a wide variety of conditions and disorders. A microstimulator usually includes or carries stimulating electrodes intended to contact the patient's tissue, but may also have electrodes coupled to the body of the device via a lead or leads. A microstimulator may have two or more electrodes. Microstimulators benefit from simplicity. Because of their small size, the microstimulator can be directly implanted at a site requiring patient therapy.

FIG. 1 illustrates an exemplary implantable microstimulator 100. As shown, the microstimulator 100 includes a power source 145 such as a battery, a programmable memory 146, electrical circuitry 144, and a coil 147. These components are housed within a capsule 202, which is usually a thin, elongated cylinder, but may also be any other shape as determined by the structure of the desired target tissue, the method of implantation, the size and location of the power source 145, and/or the number and arrangement of external electrodes 142. In some embodiments, the volume of the capsule 202 is substantially equal to or less than three cubic centimeters.

The battery 145 supplies power to the various components within the microstimulator 100, such the electrical circuitry 144 and the coil 147. The battery 145 also provides power for therapeutic stimulation current sourced or sunk from the electrodes 142. The power source 145 may be a primary battery, a rechargeable battery, a capacitor, or any other suitable power source. Systems and methods for charging a rechargeable battery 145 will be described further below.

The coil 147 is configured to receive and/or emit a magnetic field that is used to communicate with, or receive power from, one or more external devices that support the implanted microstimulator 100, examples of which will be described below. Such communication and/or power transfer may be transcutaneous as is well known.

The programmable memory 146 is used at least in part for storing one or more sets of data, including electrical stimulation parameters that are safe and efficacious for a particular medical condition and/or for a particular patient. Electrical stimulation parameters control various parameters of the stimulation current applied to a target tissue including the frequency, pulse width, amplitude, burst pattern (e.g., burst on time and burst off time), duty cycle or burst repeat interval, ramp on time and ramp off time of the stimulation current, etc.

The illustrated microstimulator 100 includes electrodes 142-1 and 142-2 on the exterior of the capsule 202. The electrodes 142 may be disposed at either end of the capsule 202 as illustrated, or placed along the length of the capsule. There may also be more than two electrodes arranged in an array along the length of the capsule. One of the electrodes 142 may be designated as a stimulating electrode, with the other acting as an indifferent electrode (reference node) used to complete a stimulation circuit, producing monopolar stimulation. Or, one electrode may act as a cathode while the other acts as an anode, producing bipolar stimulation. Electrodes 142 may alternatively be located at the ends of short, flexible leads. The use of such leads permits, among other things, electrical stimulation to be directed to targeted tissue(s) a short distance from the surgical fixation of the bulk of the device 100.

The electrical circuitry 144 produces the electrical stimulation pulses that are delivered to the target nerve via the electrodes 142. The electrical circuitry 144 may include one or more microprocessors or microcontrollers configured to decode stimulation parameters from memory 146 and generate the corresponding stimulation pulses. The electrical circuitry 144 will generally also include other circuitry such as the current source circuitry, the transmission and receiver circuitry coupled to coil 147, electrode output capacitors, etc.

The external surfaces of the microstimulator 100 are preferably composed of biocompatible materials. For example, the capsule 202 may be made of glass, ceramic, metal, or any other material that provides a hermetic package that excludes water but permits passage of the magnetic fields used to transmit data and/or power. The electrodes 142 may be made of a noble or refractory metal or compound, such as platinum, iridium, tantalum, titanium, titanium nitride, niobium or alloys of any of these, to avoid corrosion or electrolysis which could damage the surrounding tissues and the device.

The microstimulator 100 may also include one or more infusion outlets 201, which facilitate the infusion of one or more drugs into the target tissue. Alternatively, catheters may be coupled to the infusion outlets 201 to deliver the drug therapy to target tissue some distance from the body of the microstimulator 100. If the microstimulator 100 is configured to provide a drug stimulation using infusion outlets 201, the microstimulator 100 may also include a pump 149 that is configured to store and dispense the one or more drugs.

Turning to FIG. 2, the microstimulator 100 is illustrated as implanted in a patient 150, and further shown are various external components that may be used to support the implanted microstimulator 100. An external controller 155 may be used to program and test the microstimulator 100 via communication link 156. Such link 156 is generally a two-way link, such that the microstimulator 100 can report its status or various other parameters to the external controller 155. Communication on link 156 occurs via magnetic inductive coupling. Thus, when data is to be sent from the external controller 155 to the microstimulator 100, a coil 158 in the external controller 155 is excited to produce a magnetic field that comprises the link 156, which magnetic field is detected at the coil 147 in the microstimulator. Likewise, when data is to be sent from the microstimulator 100 to the external controller 155, the coil 147 is excited to produce a magnetic field that comprises the link 156, which magnetic field is detected at the coil 158 in the external controller. Typically, the magnetic field is modulated, for example with Frequency Shift Keying (FSK) modulation or the like, to encode the data. For example, data telemetry via FSK can occur around a center frequency of 125 kHz, with a 129 kHz signal representing transmission of a logic ‘1’ and 121 kHz representing a logic ‘0’.

An external charger 151 provides power used to recharge the battery 145 (FIG. 1). Such power transfer occurs by energizing the coil 157 in the external charger 151, which produces a magnetic field comprising link 152. This magnetic field 152 energizes the coil 147 through the patient 150′s tissue, and which is rectified, filtered, and used to recharge the battery 145. Link 152, like link 156, can be bidirectional to allow the microstimulator 100 to report status information back to the external charger 151. For example, once the circuitry 144 in the microstimulator 100 detects that the power source 145 is fully charged, the coil 147 can signal that fact back to the external charger 151 so that charging can cease. Charging can occur at convenient intervals for the patient 150, such as every night.

FIG. 3 shows the data telemetry circuitry in the microstimulator 100 and in the external controller 155 in further detail. Because data telemetry between these two devices along link 156 is bi-directional, each device contains both transmission circuitry (Tx) for modulating data to be telemetered, and reception circuitry (Rx) for demodulating received data. Resonant tank circuits are formed using the coils in each of the devices (157, 158) and tuning capacitors (180, 182). Values for the coils and capacitors are chosen to provide resonance in an appropriate bandwidth for communication, for example, from 120 kHz to 130 kHz to accommodate data communication at the FSK frequencies noted earlier. Switches 166 and 176 in each device couple the tank circuits to either of the transmission or reception circuits depending on whether the device is transmitting or receiving at any given moment.

Power consumption in a microstimulator 100 is preferably kept to a minimum, because lower power consumption equates to longer periods during which the microstimulator can be used to provide stimulation between charging of the battery 145 via the external charger 157. Data telemetry procedures such as those just described can affect power consumption. A microstimulator 100, regardless of whether it is currently providing stimulation to the patient, needs to be ready for the possibility that an external component, such as external controller 155, wishes to communicate with it, and hence must “listen” for relevant telemetry from the external component. Because power consumption in the external controller 155 is generally less critical (because it is external to the patient; because it can be plugged in or easily provided with fresh batteries, etc.), the external controller 155 can repeatedly broadcast its desire to communicate with the microstimulator 100, and then wait for the microstimulator 100 to telemeter an acknowledgment before sending data to the microstimulator. For example, the external controller 155 may broadcast a wake-up signal nearly continually, aside from short periods to listen for the acknowledgment from the microstimulator 100. This can be thought of as a “handshaking” or “wake up” procedure initiated by the external controller 155. The wake-up signal broadcast by the external controller 155 can comprise an alternating pattern of logic ‘1’s and ‘0’s (e.g., 0101010 . . . ). See, e.g., U.S. Patent Application Publication 2007/0049991.

This handshaking approach necessitates that the microstimulator 100, and specifically its receiver circuitry 174, be powered, because only when such circuitry 174 is powered can the microstimulator 100 recognize the wake-up signal from the external controller 155 and in turn telemeter back an acknowledgment. Ideally therefore, the receiver circuitry 174 would be powered by the microstimulator 100 at all times so that it could recognize the wake-up signal immediately. But this is not practical, especially considering the relative infrequency with which an external controller 155 might wish to communicate with a microstimulator 100. In short, keeping the receiver circuitry 174 powered at all times is not an efficient solution, as it drains too much power from the battery 145 in the microstimulator 100.

In recognition of this fact, a procedure may be employed in which the receiver circuitry 174 is only occasionally powered by the microstimulator 100, for example, once every few seconds for a window of time. While such an approach sacrifices immediacy in the microstimulator 100's recognition of the broadcast wake-up signal, it allows the receiver circuitry 174 to be powered only a fraction of the time, e.g., during a several millisecond “power-on window.” This saves power, while still allowing the external controller 155's wake-up signal to be eventually recognized and responded to by the microstimulator 100.

The problem of telemetry-based power consumption is exacerbated when more than one microstimulator 100 is implanted in a patient, as shown in FIG. 4. Use of a therapeutic network of microstimulators 100 has been discussed in the art, and is particularly useful when a patient requires relatively complicated therapy, or when therapy is appropriate within a larger portion of the patient's tissue. Although only two microstimulators 100₁ and 100₂ are shown in FIG. 4 for simplicity, it will be understood that a therapeutic network can comprise many more microstimulators.

As is known, the external controller 155 can communicate data with a particular microstimulator 100 in a network by including that microstimulator's address, e.g., [ADDR1] or [ADDR2] with the data, as shown in FIG. 4. The address is typically included in the “header” of the communication, which generally precedes the data. This allows any given microstimulator 100 to understand which communications from the external controller 155 are meant for it, and can ignore communications intended for another microstimulator 100. Such addresses may comprise several bits of data; for example each address may comprise 24 bits, divided into three 8-bit bytes in the header. This addressing scheme assumes that the microstimulators 100 have already “shaken hands” with the external controller 155 as described above: that is, the microstimulators 100 have already received the wake-up signal, and have sent acknowledgments to the external controller 155. In other words, the receiver circuits 174 in the microstimulators 100 are powered and are ready to receive communications from the external controller 155.

As just noted, an external controller 155 will typically only want to communicate with one microstimulator 100 in the network at a time. Unfortunately, all of the microstimulators 100 must power their receiver circuits 174 to listen for the external controller's wake-up signal. For example, consider an external controller 155 wishing to communicate with microstimulator 100₁. In accordance with the prior art, the external controller 155 would continually broadcast the wake-up signal, for example, 0101010 . . . as mentioned above. Both of microcontrollers 100₁ and 100₂ would have to periodically power up their receiver circuits 174 for the “power-on window”; demodulate the received wake-up signal; verify it is correct; send an acknowledgment back to the external controller 155; and then wait in a powered state for the incoming communication. Once the communication is received at both microstimulators 100, each would have to verify the address (e.g., [ADDR1]) sent with the communication. At this point, microstimulator 100₂ would recognize that the communication was not intended for it, and could power off its receiver circuitry 174.

The inventor finds this inefficient, as microstimulator 100₂ has needlessly had to power on for the window, and then further sit in a powered state to no avail. Were even more than two microstimulators 100 used in a particular therapeutic network, such needless power loss would affect that many more microstimulators.

For this reason, the inventor believes that improved methods are needed for handshaking between an external component and a plurality of microstimulators (or other medical devices) that are less wasteful of implant power, and the inventor provides solutions herein.

### BRIEF DESCRIPTION OF THE DRAWINGS

The above and other aspects of the present invention will be more apparent from the following more particular description thereof, presented in conjunction with the following drawings wherein:

FIG. 1 shows a microstimulator of the prior art.

FIG. 2 shows a microstimulator of the prior art as implanted in a patient, and in conjunction with an external controller and an external charger.

FIG. 3 shows the communication circuitry in the microstimulator and the external controller of the prior art.

FIG. 4 shows a therapeutic network of microstimulators as implanted in a patient, and in conjunction with an external controller.

FIGS. 5A-5D show a first embodiment of a wake-up procedure for a plurality of microstimulators and circuitry for implementing such procedure in the microstimulators, in which each microstimulator is assigned a unique wake-up signal to be broadcast by the external controller.

FIG. 6A-6D show a second embodiment of a wake-up procedure for a plurality of microstimulators and circuitry for implementing such procedure in the microstimulators, in which the external controller uses the microstimulators' addresses as the wake-up signal.

FIGS. 7A-7C show a third embodiment of a wake-up procedure for a plurality of microstimulators and circuitry for implementing such procedure in the microstimulators, in which non-target microstimulators can power off their receiver circuitry prematurely upon failing to verify receipt of their unique wake-up signals.

FIGS. 8A-8E show a fourth embodiment of a wake-up procedure for a plurality of microstimulators and circuitry for implementing such procedure in the microstimulators, in which non-target microstimulators can power off their receiver circuitry prematurely upon failing to verify receipt of their unique periodic wake-up signals.

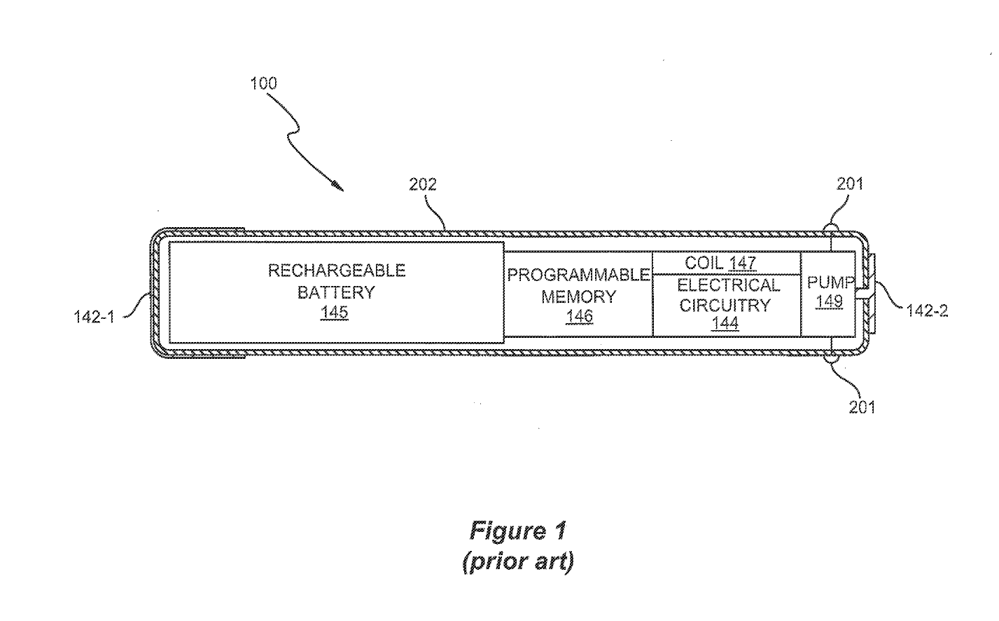

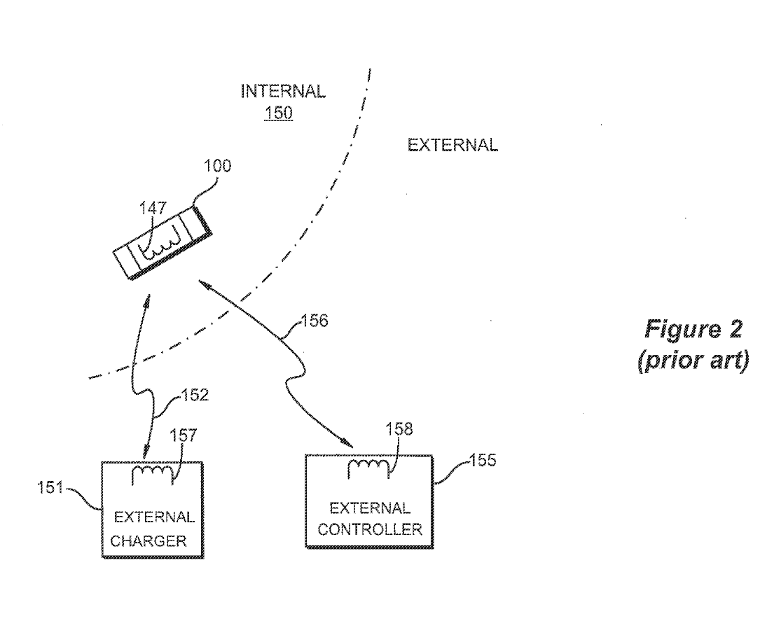

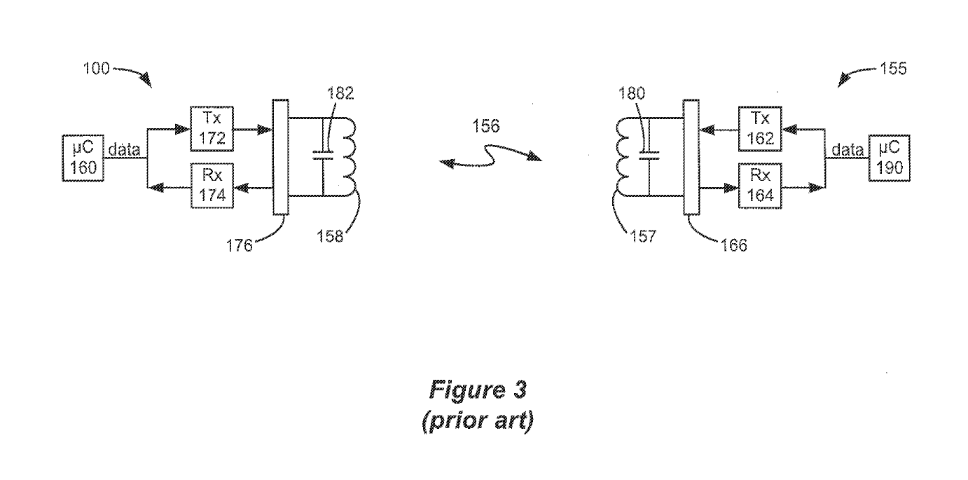

### DETAILED DESCRIPTION

In embodiments of the disclosed technique, an external controller wishing to communicate with a particular microstimulator in a microstimulator therapeutic network broadcasts a unique wake-up signal corresponding to a particular one of the microstimulators. Each microstimulator has its unique wake-up signal stored in memory, and the wake-up signals for each microstimulator are also stored in the external controller. The microstimulators power up their receiver circuits to listen for a wake-up signal at the beginning of a power-on window. Each microstimulator not recognizing the received wake-up signal (because it does not match the wake-up signal stored in its memory) will power off their receivers at the end of the power-on window, or earlier once recognition cannot be established. The one microstimulator recognizing the received wake-up signal (because it matches the wake-up signal stored in its memory) will realize that the external controller wishes to communicate with it, and will send an acknowledgment to the external controller, which will in turn send the desired communication to the now-active microstimulator. Because use of a unique wake-up signal prevents all microstimulators from waking up, power consumption (i.e., battery depletion) is minimized in the therapeutic network.

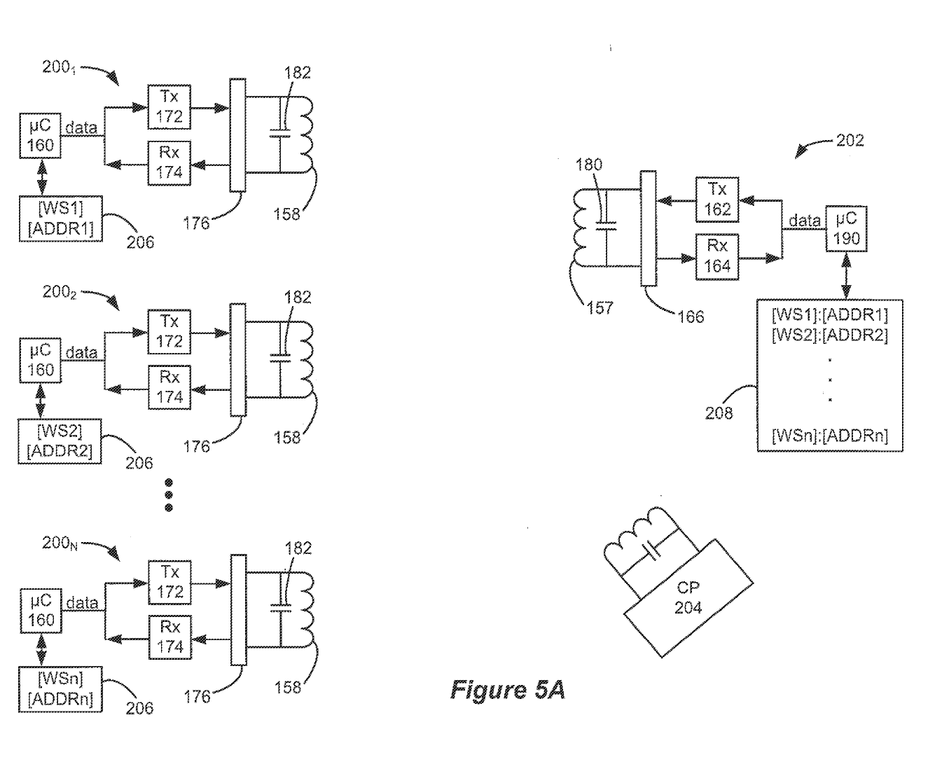

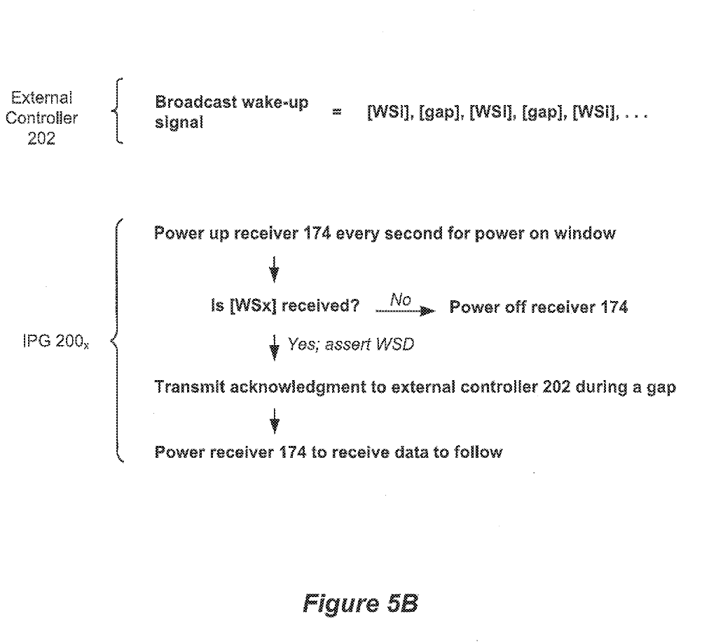

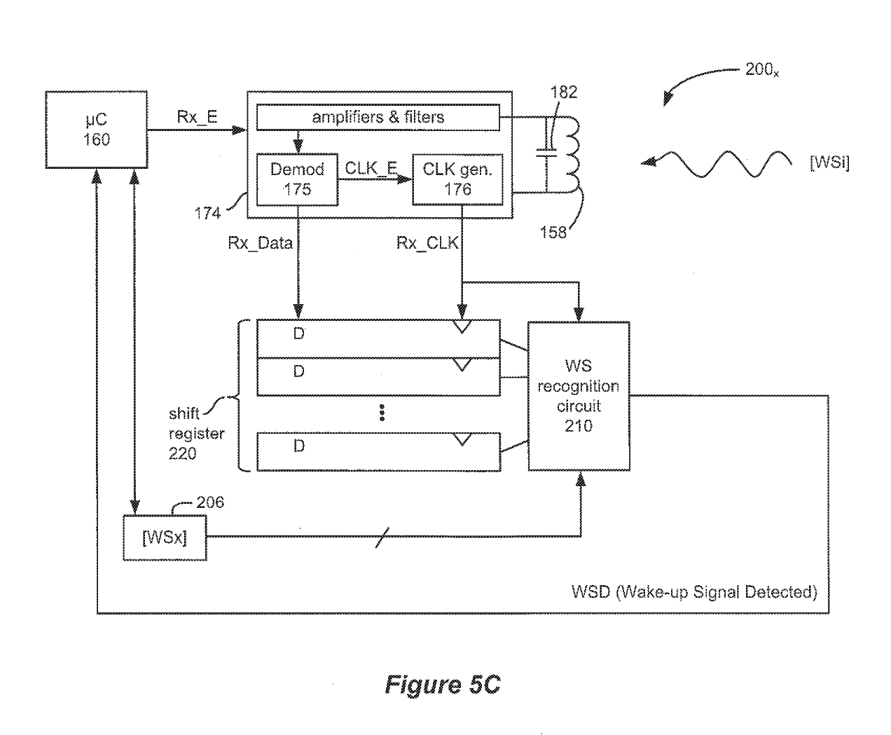

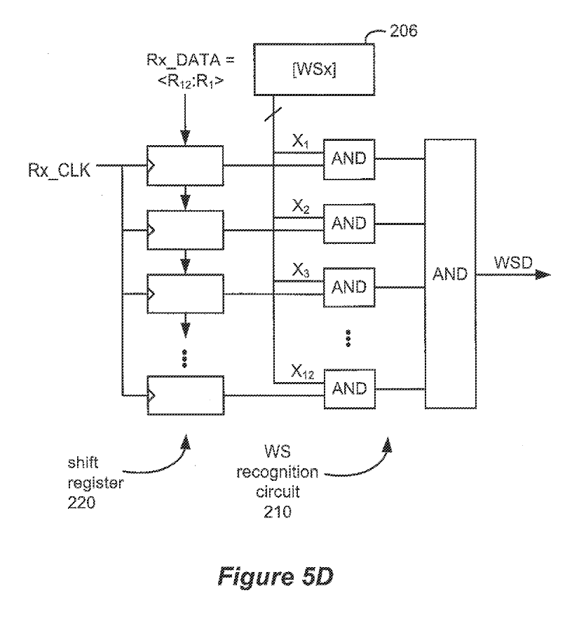

FIGS. 5A-5D show a first embodiment of the disclosed technique. Starting with FIG. 5A, a plurality of N microstimulators 200₁-200_{N} such as would be deployed in a therapeutic network in a patient are shown, as is an external controller 202 able to communicate with each.

Each microstimulator 200ₓ include a memory 206 which can be coupled to or comprises a portion of the implant's microcontroller 160. Stored in each microstimulator 200ₓ is an address ([ADDRx]) and a wake-up signal ([WSx]) that is unique to each. These addresses and wake-up signals for each microstimulator 200ₓ are also stored in a memory 208 in the external controller 202, which memory can again be coupled to or comprise a portion of the controller's microcontroller 190.

Also shown in FIG. 5A is a well-known clinician's or manufacture's programmer (CP) 204, which among functions can be used to communicate with the external controller 202 and each of the microstimulators 200ₓ. Such a CP 204 is typically used to program the external controller 202 and microstimulators 200ₓ with initialization or update data, or with new stimulation programs or settings, and as relevant here can be used to program the unique addresses and wake-up signals into each of those devices. For example, if a particular patient requires a therapeutic network of three microstimulators (200₁-200₃), the CP 204 can program a unique address and wake-up signal into microstimulator 200₁ ([ADDR1], [WS1]), microstimulator 200₂ ([ADDR2], [WS2]), and microstimulator 200₃ ([ADDR3], [WS3]), and also to program these same values into the external controller 202. Alternatively, the unique addresses of the microstimulators 200ₓ may hard-coded by the manufacturer into the software for each microstimulator 200ₓ. How the various addresses and wake up signals are loaded into the various devices is not particularly important.

Notice that each unique wake-up signal is associated with a particular microstimulator address in the memory 208 of the external controller 202 ([WSx]:[ADDRx]), such that the microcontroller 190 will know which wake-up signal to use when desiring to communicate with a particular microstimulator 200ₓ. For example, should the external controller 202 desire to communicate with microstimulator 200ᵢ—perhaps because a patient or clinician wants to change the stimulation parameters operating in that device—it would continually broadcast the wake up signal ([WSi]) that it understands to be associated with that microstimulator's address ([ADDRi]), as shown at the top of the flow chart of FIG. 5B.

Although the duration and number of bits in the wake-up signals can vary, in one example each wake-up signal ([WSx]) comprises 12 bits, each 250 microseconds in duration, although these numbers are merely exemplary. Also shown between each broadcast of the wake-up signal is a gap ([gap]) during which the external controller 202 listens for an acknowledgment from the microstimulator 200, of interest, as discussed further below. Like the wake-up signal, the gap can be of arbitrary duration, but is preferably a multiple of the of bit duration (250 μs) so as to be synchronized with the transmission of wake-up signal bits.

The bottom of the flow chart of FIG. 5B shows the operation of the microstimulators 200ₓ in the therapeutic network. As described earlier, each will power up their receivers 174 (FIG. 5A) every second or so for a power-on window duration to determine if the external controller 202 is requesting to communicate with it. Although the duration of the power-on window can vary, in one example, the power-on window is preferably longer than twice the duration of the wake-up signal plus one gap to ensure that at least one full iteration of the wake-up signal can be received and verified. For example, if the wake-up signal comprises 12 bits, and the gap comprises 3 bits, the power-on window would need to be at least 6.75 milliseconds long (250 μs*27), and preferably slightly longer to ensure the receivers 174 have sufficient time to stabilize after being powered. In this regard, note that one cannot be assured of synchronization between the start of the broadcast of the wake-up signal from the external controller 202 and the initiation of the power-on window in any particular microstimulator 200ₓ. Thus, microstimulator 200₁ may first receive the third of the 12 wake-up signal bits; microstimulator 200₂ the eighth of the 12 wake-up signal bit, etc. Dealing with this lack of synchronization, and the length of the power-on window, is discussed further below.

Continuing with FIG. 5B, at the end of the power-on window, each microstimulator 200ₓ assesses whether it has received its wake-up signal from the external controller 202. If a microstimulators 200ₓ is not able to verify receipt of its specific wake-up signal ([WSx]) as stored in its memory 206 (FIG. 5A), it powers off its receiver 174, and waits to power up again at the beginning of a next power-on window (e.g., a second later). The one microstimulator 200ₓ that does verify receipt of its wake-up signal issues a Wake-up Signal Detect (WSD) signal, which informs its microcontroller 160 of the successful detection of its wake up signal. In response to the assertion of WSD, the microcontroller 160 can then transmit an acknowledgment to the external controller 202 during a gap in the external controller 202's broadcast, and will then fully power its receiver 174 to receive the data transmission from the external controller 202 to follow.

Aspects of the flow of FIG. 5B can be implemented in software, i.e., by programming the microcontroller 160 (FIG. 5A) in each microstimulator 200ₓ. Thus, special circuitry (hardware) is not needed to implement the disclosed technique over and beyond what is typically already present in a microstimulator. However, to further understand the flow of FIG. 5B, FIGS. 5C and 5D disclose basic circuitry that can be used. An actual hardware implementation may have other circuitry features or modifications which are not noted, but would be within the purview of one skilled in the art. Again, many of the logic functions illustrated in this circuitry can be performed by the microstimulator's microcontroller 160.

In FIG. 5C, the receiver 174 is enabled (e.g., powered) by signal Rx_E issued from the microcontroller 160. This enable signal Rx_E will be periodically asserted to initiate the power-on window during which the microstimulator 200ₓ will listen for the broadcast wake-up signal [WSi] (if any) being broadcast from the external controller 202. As noted earlier, the wake-up signal can be modulated using a suitable protocol, such as FSK, in which each logic state in the wake-up signal is represented by a particular frequency, such as a 129 kHz signal representing transmission of a logic ‘1’ and 121 kHz representing a logic ‘0’. These frequencies cause the microstimulators' tank circuit (182/158) to resonate, and the received signal is amplified and filtered as necessary, and eventually demodulated (175) back into a digital stream of data bits, Rx_Data.

Additionally, demodulator 175 asserts a clock enable signal, CLK_E, to clock generation circuitry 176. Clock enable signal CLK_E is asserted by demodulator 175 immediately upon sensing resonance after a period of no resonance, i.e., upon demodulating the first bit in the broadcast wake-up signal after a gap period. The clock issued by the clock generator 176, Rx_CLK, in response to CLK_E will have the same period of the transmitted data (i.e., 250 μs) and will have as many cycles as there are bits of data in the wake-up signal, for example 12 cycles to continue the example above. Note that this clocking scheme—which generates a clock Rx_CLK only after receipt of data following a gap—addresses the lack of synchronicity between the external controller 202 and the microstimulators 200ₓ discussed above.

The received bits of the wake-up signal, RX Data, are loaded into a shift register 220 under control of the recovered clock, Rx_CLK. In this embodiment, the shift register 220 has as many registers (e.g., 12) as there are bits in the wake-up signal. The first cycle of Rx CLK will load the most significant bit of the received wake-up signal (R₁₂) into the first register in shift register 220, as shown in further detail in FIG. 5D, with subsequent clock cycles moving other bits in the received wake-up signal through the shift register. After the clock generator 176 has output its last (12^{th}) clock cycle, the entire wake-up signal will have been entered into the shift register 220.

As noted earlier, the power-on window in this example needs to be asserted for at least twice the duration of wake-up signal plus the gap to ensure that the wake-up signal is fully captured. Assume for example a worst case in which the power-on window is asserted upon the arrival of the first (most-significant) bit of the wake-up signal broadcast from the external controller 202. In this instance, because the demodulator 175 has not yet received a gap (no modulation condition), the clock generator will not generate clock Rx CLK, and this first bit of the wake-up signal will not be loaded into the shift register 220, and neither will any subsequent bits. Instead, the demodulator 175 must wait for the gap, then assert the clock to capture the next broadcast of the wake-up signal. In sum, this worst-case example requires the power-on window to extend for an entire wake-up signal broadcast which is not captured, followed by a gap, and followed by the next broadcast wake-up signal, thus arriving at the minimal power-on window duration just discussed. However, in other embodiments, the power-on window can be shortened for even greater power savings, although this may require modification to the clock generation circuitry 176 and to WS recognition circuit 210 discussed in the next paragraph.

Once the received wake-up signal is fully loaded in this fashion into the shift register 220, it is compared to the wake-up signal (WSx) stored in memory 206 in each microstimulator 200ₖ using Wake-up Signal (WS) recognition circuitry 210. WS recognition circuitry 210 is represented in FIG. 5D by a series of AND gates, each comparing corresponding bits in the stored wake-up signal (Xᵢ) and the received wake-up signal (Rᵢ) as latched in the shift register 220. If all bits match as determined by the final AND gate in FIG. 5D, the WS recognition circuit 210 issues the Wake-up Signal Detected (WSD) signal. As a reminder, only one microstimulator 202ₓ in the therapeutic network will verify a match between the received wake-up signal and its stored wake-up signal, and thus only one will assert WSD. For those microstimulators 200ₓ not issuing WSD, their microcontrollers 160 will disable the receiver enable signal (Rx_E) at the end of the power-on window, thus powering down the receiver 174 until the next power-on window is initiated (e.g., a second or so later).

Referring again to FIG. 5C, once the WSD signal is received by the microcontroller 160 of the microstimulator 200ₓ of interest, the microcontroller 160 will prepare the microstimulator 200ₖ for communications with the external controller 202. First, the microcontroller 160 will activate its transmitter 172 (FIG. 5A) to send an acknowledgment to the external controller 202. Preferably, but not necessarily, transmission of the acknowledgement can occur during a gap in the external controller 202's broadcast of the wake-up signal. Because WSD is asserted at the end of receipt of a wake-up signal, and hence at the beginning of a gap, such acknowledgement broadcast can essentially begin immediately upon assertion of the WSD signal, although perhaps some time will be necessary to initialize the transmitter 172.

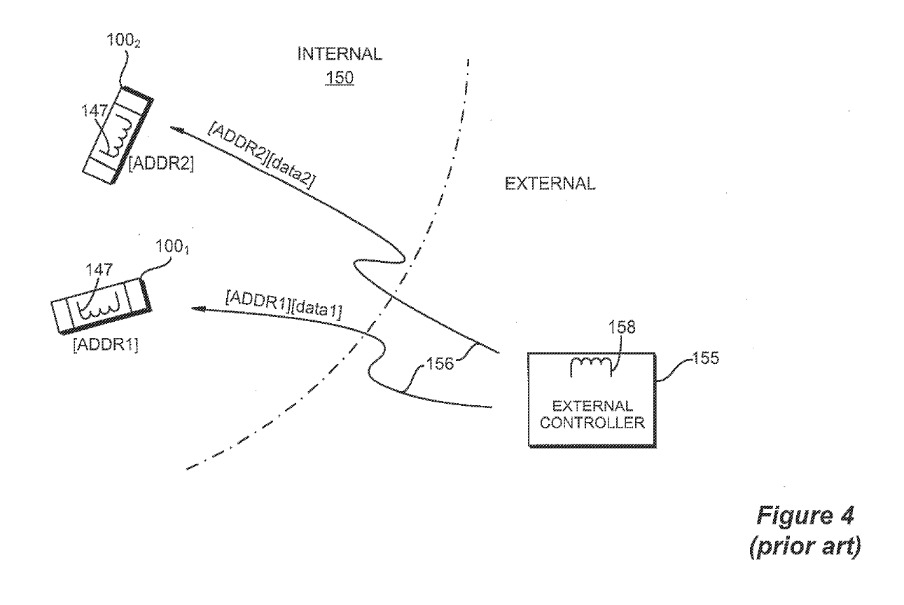

Thereafter, the microcontroller 160 in the microstimulator 200ₓ of interest prepares for communications with the external controller 202, e.g., by asserting (or continuing to assert) enable signal Rx_E to keep the receiver 174 powered to receive the external controller 202's data transmission. Thereafter, communications between the microstimulator of interest 200ₓ and the external controller 202 can occur as normal, with the external controller 202 sending data to the microstimulator 200ₓ using the header (addressing) scheme discussed earlier (see FIG. 4). Note that the microstimulator 200ₓ can in return provide the external controller 202's address in its communication with the external controller 202, which ensures that the proper external device will receive the microstimulator 200ₓ's communication. (More than one external device, such as the clinician/manufacturer's programmer CP 204 (FIG. 5A), can also communicate bi-directionally with the microstimulators 200ₓ, and therefore each microstimulator 200ₓ preferably includes the address of the relevant external device to ensure its data arrives at the right location). However, such addressing of the external controller (or other external devices) is not shown for clarity.

With the technique of FIGS. 5A-5D fully explained, its benefits can be appreciated. As discussed in the Background, in the prior art, all microstimulators 200ₓ in a therapeutic network would respond to a common wake-up signal (e.g., 101010101010), and hence all would have their receivers 174 powered up and ready for an incoming data transmission from the external controller 202, even though only one of the microstimulators 200ₓ is intended as the target for that transmission. Each non-target microstimulator 200ₓ would needlessly have to then demodulate the header of the incoming transmission, including the address, to decide whether the transmission was intended for it, and then power off once the address was not recognized. Thus, in sum, each non-target microstimulator 200ₓ would have to power up its receiver 174 for at least the duration of the wake-up signal and the duration of the address in the header. If a 12-bit wake-up signal and a 24-bit address is used, and assuming a 250 μs duration of the bits, this means that each non-target microstimulator 200ₓ would need to be powered for 9 milliseconds ((12+24)*250 μs). By contrast, the disclosed technique allows the non-targeted microstimulators 200ₓ to power for only 6.75 milliseconds, as discussed above. This power savings in each non-target microstimulator 200ₓ is significant, and marks a particularly significant savings in the therapeutic network as a whole as the number of microstimulators 200ₓ increases.

Additionally, such power savings can be further improved by reducing the number of bits of each unique wake-up signal. For example, if an eight bit wake-up signal is used, each non-target microstimulator 200ₓ would only need to power on for 4.75 milliseconds before recognizing that an incoming transmission was not intended for it. In this regard, note that the number of bits in the unique wake-up signals is driven by the number of microstimulators 200ₓ in each therapeutic network, i.e., in each patient. As the number of microstimulators 200ₓ in any given patient may be relatively small, the number of required bits in each unique wake-up signal may likewise be relatively small. For example, a network of 16 microstimulators 200ₓ would require only four bits to encode 16 unique wake-up signals (from 0000 to 1111), which would reduce power consumption in the non-target microstimulators 200ₓ even further to 2.75 milliseconds. That being said, it may be desired to use unique wake-up signals having more than the minimum number of bits to improve reliability in the receipt of such signals. Note that if the number of bits in the wake-up signal is reduced, the number of registers in the shift register 200, the number of clock cycles in Rx_CLK, etc., can be reduced as well.

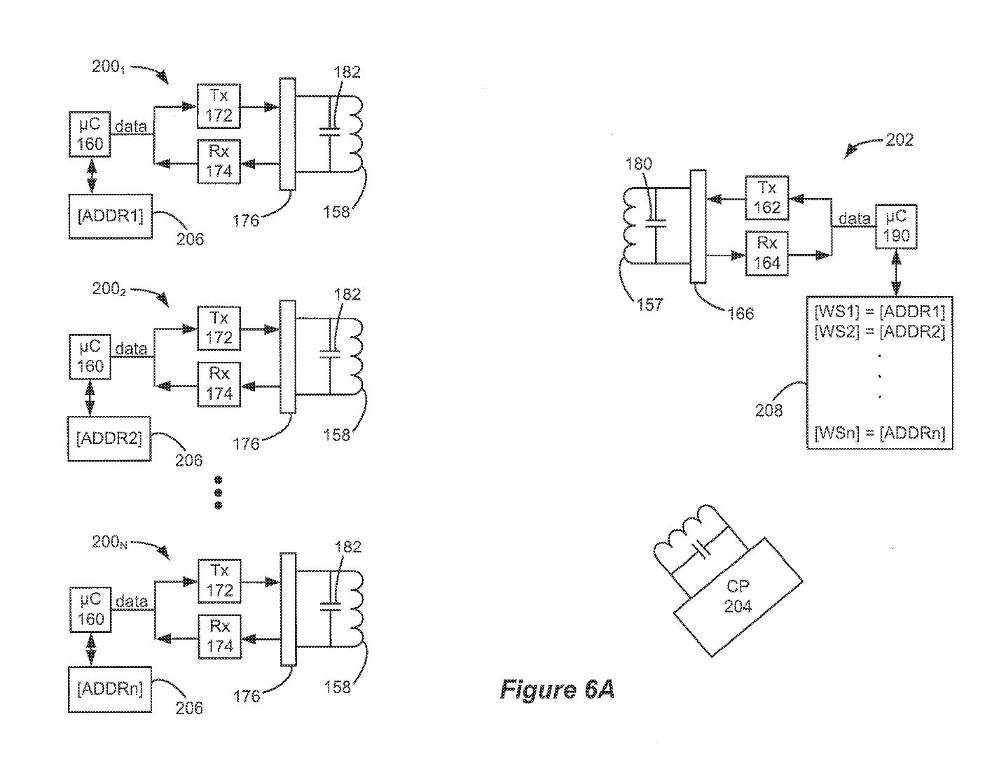

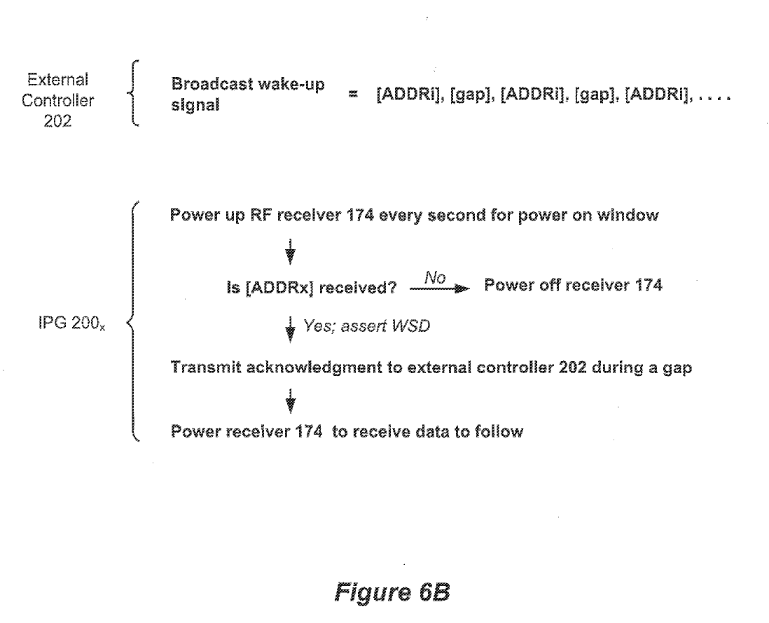

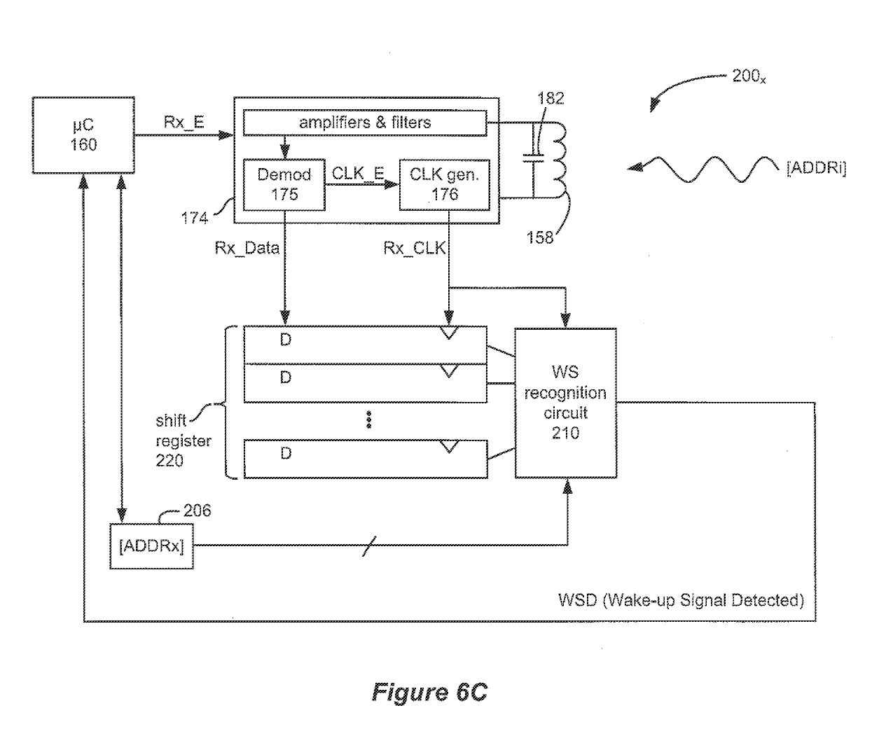

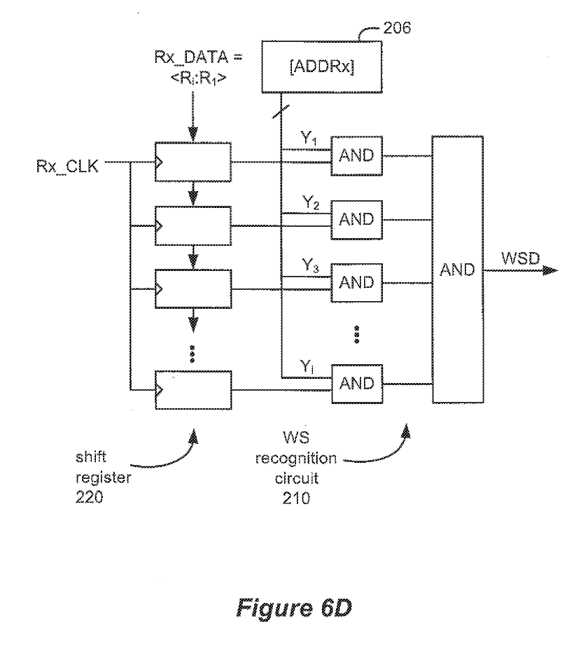

FIGS. 6A-6D illustrate another embodiment of the disclosed technique in which the microstimulator 200ₓ's addresses ([ADDRx]) are used as the unique wake-up signal, as well as to send data to a particular microstimulator once handshaking has occurred. This embodiment recognizes that unique wake-up signals do not have to be used if data already uniquely identifying particular microstimulators 200ₓ in a therapeutic network are already established. This modification may not reduce the power of the wake-up procedure in the therapeutic network depending on its particular implementation, but would be indicated in applications where it is desired to simplify the wake-up procedure by using already-existing microstimulator addresses.

FIG. 6A show the microstimulators 200ₓ, the external controller 202, and the clinician/manufacturer programmer 204, and shows the unique addresses for each microstimulator ([ADDRx]) stored in their memories 206 and in the memory 208 of the external controller 202. Because such data is stored in this manner in a traditional microstimulator system, this embodiment does not require additional system preparation other than to program the microstimulators to use their stored addresses during the wake-up procedure. Again, the external controller 202 will use the microstimulators' address as the wake-up signal, which is represented in memory 208 as [WSx]=[ADDRx].

FIG. 6B shows the wake-up procedure operating in the external controller 202 and each of the microstimulators 200ₓ. However, when the external controller desires to communicate with a particular microstimulator 200ᵢ, it will continually broadcast that microstimulator's address ([ADDRi]), with gaps between the broadcast of each address as before. Each microstimulator 200ₓ as before periodically powers on its receiver 174 to establish a power-on window, and determines whether its address has been received. If not, it powers off its receiver 174 until the next power-on window. If so, i.e., if the received wake-up signal ([ADDRi]) matches the address stored in its memory, it transmits an acknowledgement to the external controller 202, and power (or continues to power) its receiver 174 to receive the communications to follow. Again, such future communications will include in its header the same address used to “wake up” the microstimulator 200ᵢ of interest in this embodiment.

The circuitry in FIGS. 6C and 6D are modified compared to their counterparts of FIGS. 5C and 5D to account for use of the microstimulator's address as the wake-up signal. Thus, as shown in FIG. 6C, memory 206 provides the microstimulators' address ([ADDRx]) to the WS recognition circuit 210. The shift register 220 latches the received wake-up signal ([ADDRi]) as before, although perhaps with modifications if the addresses are of different lengths from the 12-bit wake-up signal considered earlier. For example, if the addresses are 24-bits, then shift register 220 would include 24 registers, Rx_CLK would provide 24 clock cycles, etc. In any event, the wake-up signal is captured in the shift register 220 as Rx Data as shown in FIG. 6D, and bits of the stored microcontroller address (Yᵢ) are compared to corresponding bits in the received address (R) in the WS recognition circuit 210. In the event of a match, WSD is asserted, and the microcontroller 200ₓ prepares for communication with the external controller as already discussed. If not, Rx_E is disabled until the next power-on window.

Embodiments of the disclosed technique thus far has required receipt and verification of the entire wake-up signal at the microstimulators 200ₓ, and as such have required the microstimulators 200ₓ to power their receivers 174 for the entirety of the power-on window. However, this is not strictly required, and in other embodiments only a portion of the wake-up signal needs to be received for the microstimulators 200ₓ to verify receipt of their unique wake-up signal. When a non-target microstimulator 200ₓ cannot verify a portion of the wake-up signal in a portion of its power-on window, it prematurely powers off its receiver 174 before the expiration of the power-on window to save power. Such embodiments are discussed subsequently.

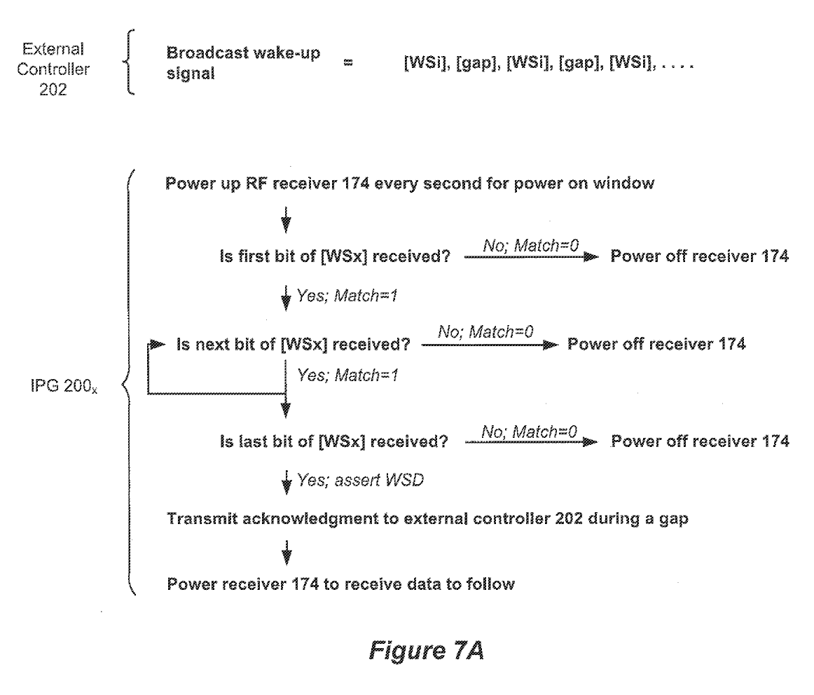

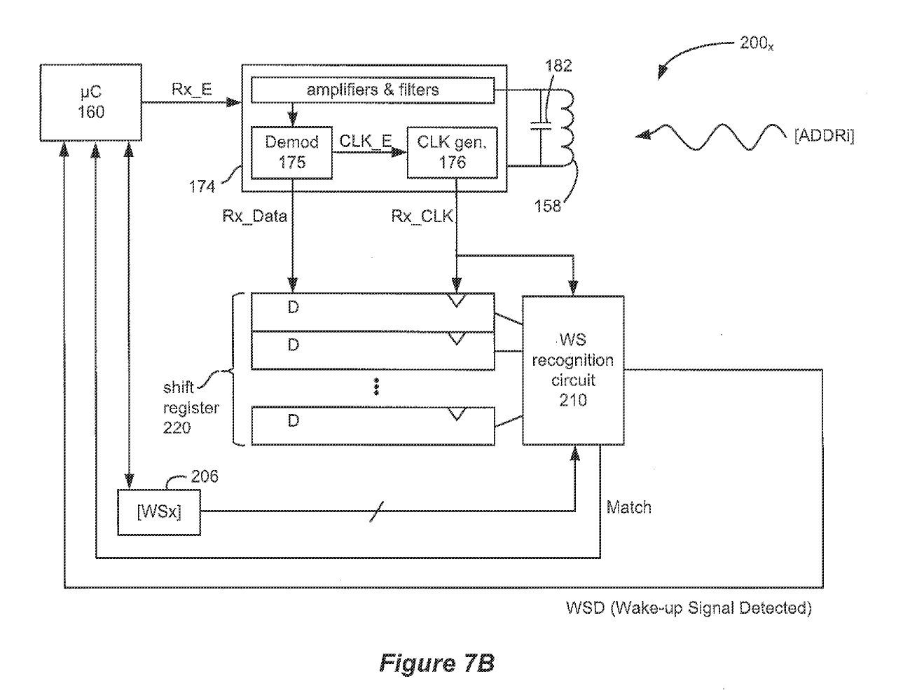

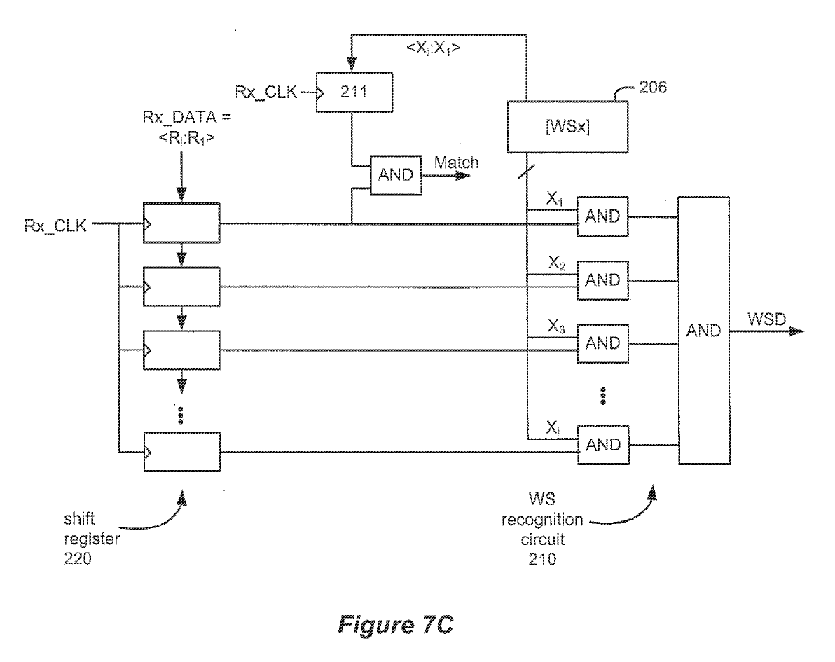

In FIGS. 7A-7C, the wake-up recognition circuitry 210 (FIGS. 7C) is modified to issue an additional signal “Match” which results from an assessment of each bit of the received wake-up signal. In this embodiment, it is assumed that the wake-up signal [WSi] broadcast by the external controller 202 is different from the unique addresses for the microstimulators 200ₓ, and so is similar to the example of FIGS. 5A-5D. However, microstimulator addresses could also be used for the wake-up signals as in FIGS. 6A-6D.

In FIG. 7A, each bit of the received wake-up signal [WSi] is assessed after it demodulated, and compared with the corresponding bit of the unique wake-up signals [WSx] stored in memory 206 in each of the microstimulators 200ₓ. If the first (most-significant) bits match, Match is asserted (Match=1), and the next-most significant bits in the received and stored wake-up signals are compared, and so on until all bits have been compared. If at any time any of the corresponding bits in the received and stored wake-up signals do not match, Match is disabled (Match=0), which informs the microcontroller 160 to immediately disable the receiver 174 via signal Rx_E. This can occur at any time during the power-on window, and so the receiver 174 may only be powered for a portion of the window, as mentioned earlier. Once its receiver 174 is disabled, the affected non-target microstimulator 200ₓ will not again enable its receiver until the next power-on window. If all of the bits match, WSD is asserted as before, and the affected target microstimulator 200ₓ issues an acknowledgment to the external controller 202 and powers its receiver 174 to receive the data to follow.

FIG. 7B and 7C show circuitry for implementing the wake-up procedure of FIG. 7A. Notice in FIG. 7B that Wake-up Signal (WS) recognition circuit 210 issues signal Match in addition to WSD, which signals were referred to above. FIG. 7C shows further details concerning the generation of signal Match. (Circuitry for generating signal WSD remains unchanged from FIG. 5D for example, and is not again explained). New to FIG. 7C is the addition of a latch 211, which under control of RX CLK serially captures the bits <Xᵢ:Xᵢ> of the wake-up signal stored in memory 206. In reality, a latch is not necessary, and instead the memory 206 can be controlled to simply output the bits in synchronization with the clock Rx_CLK. The stored bits <Xᵢ:Xᵢ> are serially compared with corresponding bits in the received wake-up signal <Rᵢ:Rᵢ> by taking the output of the first register in shifter register 220, such that X, is compared with Rᵢ during the first clock cycle, then Xᵢ₋₁ with Rᵢ₋₁ the next clock cycle, etc. If at any time such corresponding bits do not match, signal Match, output by an AND gate, will equal zero. When this condition is received at the microcontroller 160 (FIG. 7B), microcontroller 160 will disable Rx_E, which will power off the receiver 174 and prevent the demodulation of any further bits in the broadcasted wake-up signal [WSi] during the power-on window. In effect the power-on windows in the non-target microstimulators 200ₓ are cut short by condition Match=0. Only when Match=1 and WSD=1 at the end of the power-on window will the microcontroller 160 in the target microstimulator 200ₓ understand that its entire, unique wake-up signal has been received, and prepare that microstimulator for the incoming communication from the external controller 202 to follow.

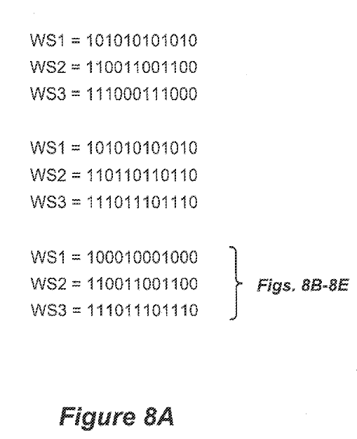

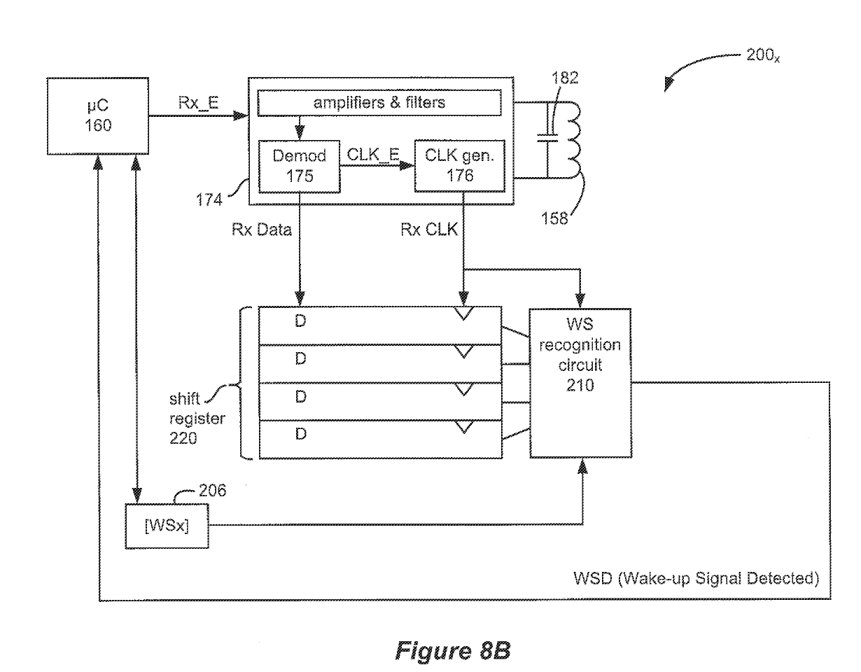

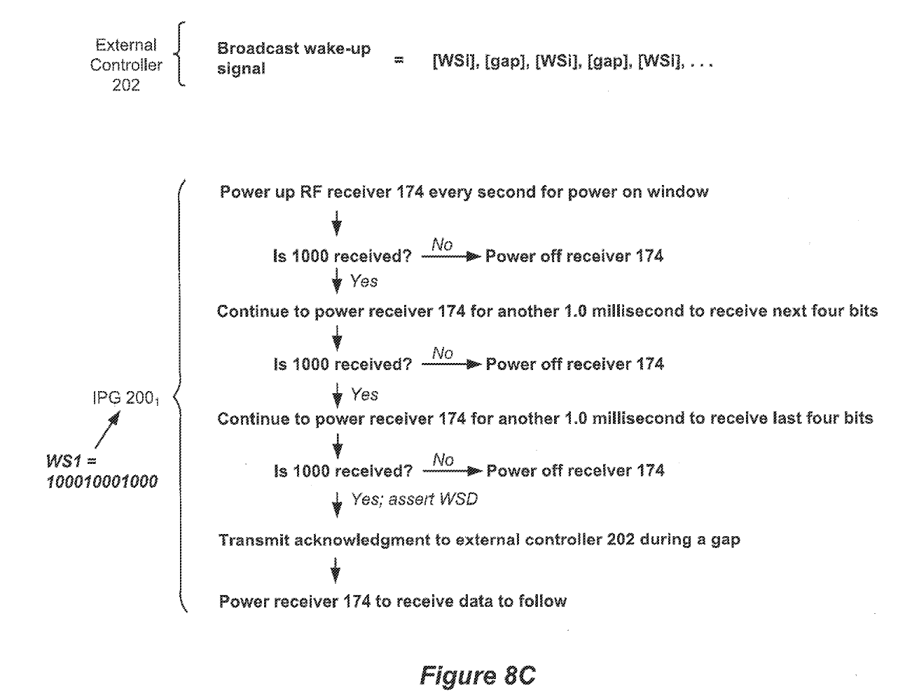

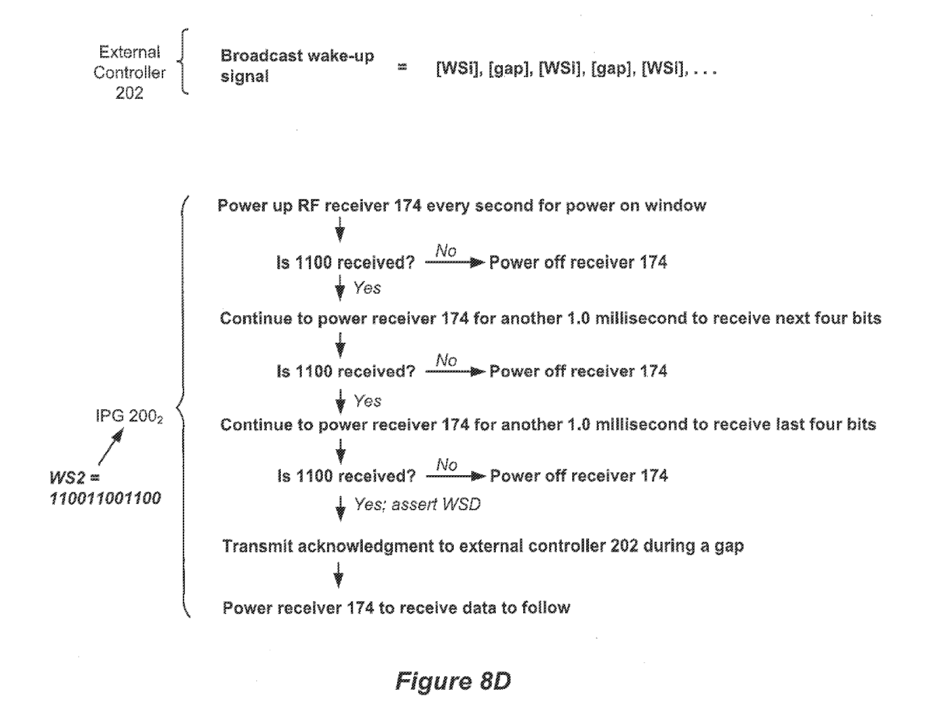

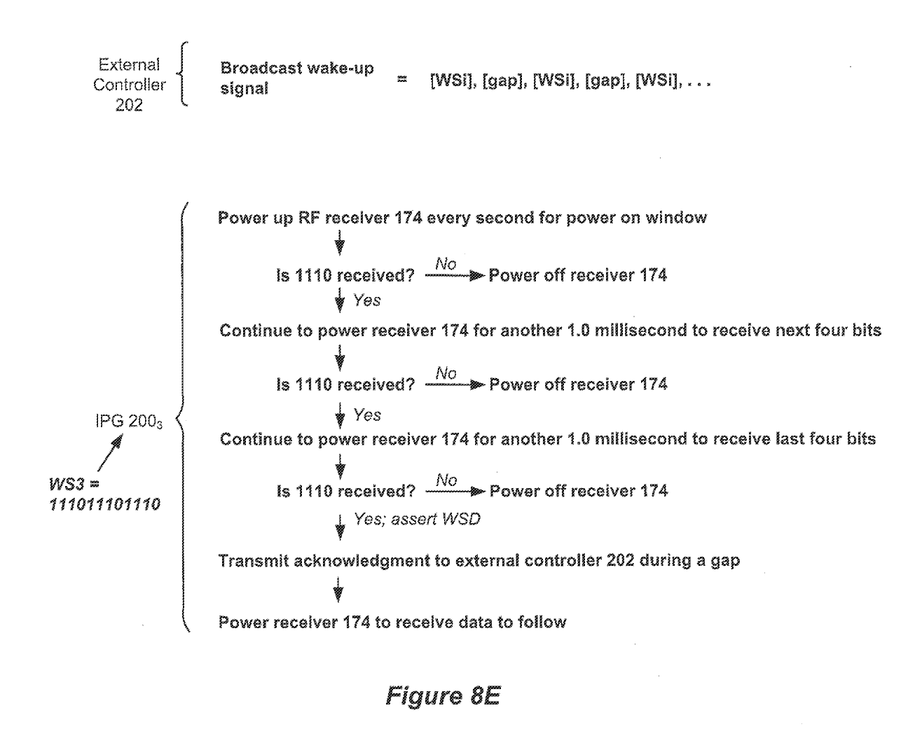

FIGS. 8A-8E illustrate another embodiment in which non-target microstimulators 200ₓ can power down their receivers 174 early in the power-on window. FIG. 8A shows three examples of unique wake-up signals ([WS1], [WS2], [WS3]) that can be used in a simple network comprising three microstimulators 200₁, 200₂, and 200₃. As in FIG. 5A, these wake-up signals are stored in the microstimulators 200ₓ and the external controller 202 and are associated with each microstimulator 200ₓ's address in the external controller 202, although this is not again depicted.

Each of the unique 12-bit wake-up signals in FIG. 8A are periodic, and have a 4-bit portion of bits that repeats three times in each. In the bottom example, which is illustrated in further detail in FIGS. 8B-8E, the first wake-up signal ([WS1]) repeats the sequence 1000; the second wake-up signal ([WS2]) 1100, and the third wake-up signal ([WS3]) 1110. Such simple periodic signals provide simple examples for illustration of the technique, but are not strictly required.

FIG. 8B shows circuitry for implementing the wake-up procedure using the wake-up signals of FIG. 8A. In FIG. 8B, notice that the shift register 220 contains a fewer number of registers matching the periodicity of the bits (four) in the wake-up signals. This allows four-bit portions of the 12-bit wake-up signals to be assessed in series, with the receiver 174 being powered down should any of the portions not match the unique wake-up signals stored. If the first four bits of the received wake-up signal match the first four bits of the wake-up signal stored memory 206, the receiver continues to be powered, and a next four received bits are assessed; if not the receiver 174 is powered off at the end of the four bit portion, i.e., early in the power-on window. If the next four bits match, the receiver 174 continues to be powered to receive the last four bits; if not, the receiver 174 is powered off. If the last four bits match, then WSD is asserted and the microstimulator prepares for the incoming communication from the external controller 202 as before. The operation of these flows in each of the example three microstimulators 200₁, 200₂, and 200₃ are illustrated in FIGS. 8C-8E respectively. As with the embodiment of FIGS. 7A-7C, each non-target microstimulators 200ₓ conserves power by powering off its receivers 174 early in the power-on window once it is clear that its corresponding wake-up signal has not been received from the external controller 202.

While the invention herein disclosed has been described by means of specific embodiments and applications thereof, numerous modifications and variations could be made thereto by those skilled in the art without departing from the literal and equivalent scope of the invention set forth in the claims.

What is claimed is:

1. A method for communicating with an implantable medical device in a therapeutic network comprising a plurality of implantable medical devices, comprising:
broadcasting a wake-up signal from an external device desiring to send a communication to a first of the implantable medical devices, wherein the wake-up signal corresponds to the first implantable medical device;
powering a receiving circuit in each of the implantable medical devices to receive the wake-up signal at each implantable medical device;
assessing the validity of the wake-up signal at each implantable medical device;
if at least a portion of the wake-up signal is assessed as valid at a given implantable medical device, sending an acknowledgment from that implantable medical device to the external device, and thereafter receiving at that implantable medical device the communication from the external device; and
if at least a portion of the wake-up signal is not assessed as valid at a given implantable medical device, powering off the receiving circuit at that implantable medical device.

2. The method of claim 1, wherein the wake-up signal comprises an address for the first implantable medical device.

3. The method of claim 2, wherein the communication includes the address for the first implantable medical device.

4. The method of claim 1, wherein the wake-up signal is different from an address for the first implantable medical device included in the communication.

5. The method of claim 1, wherein powering the receiving circuit in each of the implantable medical devices comprises powering the receiving circuitry for a power-on window.

6. The method of claim 1, wherein powering the receiving circuit in each of the implantable medical devices comprises periodically powering the receiving circuitry for a power-on window.

7. The method of claim 1, wherein powering the receiving circuit in each of the implantable medical devices is not synchronized with the broadcasting of the wake-up signal.

8. The method of claim 1, wherein the receiving circuits in the implantable medical devices are not powered at the same time.

9. The method of claim 1, wherein the wake-up signal is broadcast continuously.

10. The method of claim 9, wherein the continuous broadcast of the wake-up signal contains gaps for receiving the acknowledgment.

11. The method of claim 1, wherein each of the implantable medical devices has a unique wake-up signal, wherein each unique wake-up signal is stored in a memory in the external device, and wherein broadcasting the wake-up signal from an external device comprises reading the unique wake-up signal for the first implantable medical device from the memory.

12. A method for communicating with an implantable medical device in a therapeutic network comprising a plurality of implantable medical devices, comprising:
broadcasting a wake-up signal from an external device desiring to send a communication to a first of the implantable medical devices, wherein the wake-up signal corresponds to the first implantable medical device;
powering a receiving circuit in each of the implantable medical devices to receive a first portion of the wake-up signal at each implantable medical device;
assessing the validity of the first portion at each implantable medical device;
if the first portion is assessed as valid at a given implantable medical device, continuing to power the receiver circuit at that implantable medical device to receive at least a second portion of the wake-up signal; and
if the first portion is not assessed as valid at a given implantable medical device, powering off the receiving circuit at that implantable medical device.

13. The method of claim 12, further comprising if the second portion is assessed as valid at a given implantable medical device, continuing to power the receiver circuit at that implantable medical device to receive at least a third portion of the wake-up signal, and if the second portion is not assessed as valid at that given implantable medical device, powering off the receiving circuit at that implantable medical device.

14. The method of claim 12, further comprising if the entire wake-up signal is assessed as valid at a given implantable medical device, sending an acknowledgment from that implantable medical device to the external device, and thereafter receiving at that implantable medical device the communication from the external device.

15. The method of claim 12, wherein the wake-up signal comprises an address for the first implantable medical device.

16. The method of claim 15, wherein the communication includes the address for the first implantable medical device.

17. The method of claim 12, wherein the wake-up signal is different from an address for the first implantable medical device included in the communication.

18. The method of claim 12, wherein powering the receiving circuit in each of the implantable medical devices comprises powering the receiving circuitry at the beginning of a power-on window.

19. The method of claim 12, wherein powering the receiving circuit in each of the implantable medical devices comprises periodically powering the receiving circuitry at the beginning of a power-on window.

20. The method of claim 12, wherein powering the receiving circuit in each of the implantable medical devices is not synchronized with the broadcasting of the wake-up signal.

21. The method of claim 12, wherein the receiving circuits in the implantable medical devices are not powered at the same time.

22. The method of claim 12, wherein the wake-up signal is broadcast continuously.

23. The method of claim 22, wherein the continuous broadcast of the wake-up signal contains gaps for receiving the acknowledgment.

24. The method of claim 12, wherein each of the implantable medical devices has a unique wake-up signal, wherein each unique wake-up signal is stored in a memory in the external device, and wherein broadcasting the wake-up signal from an external device comprises reading the unique wake-up signal for the first implantable medical device from the memory.

25. The method of claim 12, wherein the first and second portions comprise single bits.

26. The method of claim 12, wherein the wake-up signal comprises equal periodic portions, and wherein each of the first and second portions comprises the periodic portion.

27. An external device for communicating with a plurality of implantable medical devices, comprising:
controller circuitry;
memory coupled to or comprising part of the controller circuitry, wherein the memory comprises a unique wake-up signal and an associated unique address for each of the plurality of implantable medical devices;
a transmitter coupled to the controller circuitry, wherein the transmitter is configured to transmit from the memory a first of the plurality of wake-up signals corresponding to a selected one of the plurality of implantable medical devices; and
a receiver, wherein the receiver is configured to receive an acknowledgment from the selected implementable medical device,
wherein the transmitter is further configured after receipt of the acknowledgment to transmit data from the controller circuitry to the selected implantable medical device, wherein the data is accompanied by a first of the plurality of addresses associated with the first wake-up signal.

28. The external device of claim 27, wherein the first wake-up signal is transmitted continuously.

29. The external device of claim 28, wherein the continuous transmission of the first wake-up signal contains gaps, and wherein the receiver is further configured to receive the acknowledgment during one of the gaps.

30. The external device of claim 27, wherein the transmitter and receiver are coupled to a resonant tank circuit.

31. The external device of claim 30, wherein the transmitter and receiver are coupled to the resonating tank circuit by a switch, wherein the switch couples either the transmitter or the receiver to the resonant tank circuit at any given time.

32. The external device of claim 30, wherein the tank circuit comprises a coil and a capacitor.

33. The external device of claim 27, wherein the transmitter and receiver operate in accordance with a Frequency Shift Keying protocol.

34. An external device for communicating with a plurality of implantable medical devices, comprising:
controller circuitry;
memory coupled to or comprising part of the controller circuitry, wherein the memory comprises a unique address for each of the plurality of implantable medical devices;
a transmitter coupled to the controller circuitry, wherein the transmitter is configured to transmit from the memory a first of the plurality of addresses corresponding to a selected one of the plurality of implantable medical devices; and
a receiver, wherein the receiver is configured to receive an acknowledgment from the selected implementable medical device,
wherein the transmitter is further configured after receipt of the acknowledgment to transmit data from the controller circuitry to the selected implantable medical device, wherein the data is accompanied by the first address.

35. The external device of claim 34, wherein the first address is transmitted continuously.

36. The external device of claim 35, wherein the continuous transmission of the first address contains gaps, and wherein the receiver is further configured to receive the acknowledgment during one of the gaps.

37. The external device of claim 34, wherein the transmitter and receiver are coupled to a resonant tank circuit.

38. The external device of claim 37, wherein the transmitter and receiver are coupled to the resonating tank circuit by a switch, wherein the switch couples either the transmitter or the receiver to the resonant tank circuit at any given time.

39. The external device of claim 37, wherein the tank circuit comprises a coil and a capacitor.

40. The external device of claim 34, wherein the transmitter and receiver operate in accordance with a Frequency Shift Keying protocol.
